(19) United States
(12) Patent Application Publication
(10) Pub. No.: US 2013/0046281 A1
(43) Pub. Date: Feb. 21, 2013

(54) Closed Loop Infusion Formulation Delivery System

(71) Applicant: Jonathan C. Javitt, Chevy Chase, MD (US)

(72) Inventor: Jonathan C. Javitt, Chevy Chase, MD (US)

(21) Appl. No.: 13/212,171

(22) Filed: Aug. 17, 2011

(51) Int. Cl. A61M 5/168

(52) U.S. Cl. 604/504; 604/66; 604/503

(57) ABSTRACT

A closed loop infusion formulation delivery system for controlling a biological state in the body of a user includes a sensor for generating a signal representative of measured parameters and a computing element for processing the generated signal. The computing element adjusts control parameters within an algorithm, calculates a delivery rate of an infusion formulation and generates commands based on the calculated delivery rate. A remotely located monitoring station communicates with the computing element via radio or wire to further adjust the control parameters within the algorithm to meet changing needs in the body of the user.

### BACKGROUND OF THE INVENTION

1. Field of the Invention

The present invention relates generally, to apparatus and method of managing the delivery of infusion formulations to a person and, more particularly to an external monitor having automated and human elements which oversees and adjusts a closed loop algorithm and system for accurately regulating the delivery rate of an infusion formula such as insulin to a person and which may be used to protect a patient's safety by providing an opportunity to manually alter infusion flow, shut off infusion flow, potentially activate flow of a second, rescue drug, communicate with the patient, or in an emergency situation geolocate the patient and provide an emergency response.

2. Description of Related Art

As disclosed in prior art U.S. Pat. No. 6,740,072, the contents of which are incorporated herein by reference in its entirety, and where portions of the description of the prior art patent are reproduced herein in whole or in part to provide an understanding of this invention, infusion formulation delivery device such as an infusion pump has been used for the programmed delivery of measured doses of an infusion formulation, here defined as the substance being delivered by the infusion pump. This substance may comprise either a mixture of different components or it may be a single, pure substance, including, but not limited to drugs, dyes or other indicators, nutrient, or the like. A typical example of such use is the delivery of an insulin formulation to a patient.

In the case where the infusion formulation is an insulin formulation, a sensing device may regulate the delivery of the insulin formulation by sensing the levels of blood glucose in the person. The delivery of the insulin formulation may be controlled by a control device associated with the pump having as an input a sensed blood glucose level. The control device may control activation of the pump to deliver an appropriate amount of the insulin formulation in accordance with the sensed blood glucose level.

Insulin is a peptide hormone normally formed within the human pancreas. Because it regulates carbohydrate (sugar) metabolism, insulin is required for normal metabolic function. More specifically, insulin helps the body metabolize glucose. To avoid medical problems such as hypoglycemia and hyperglycemia, blood glucose levels should be maintained within a specific range. A normal range for glucose in the human body may be between 85 and 120 milligrams/deciliter (mg/dl).

In a non-diabetic person, insulin is secreted by the pancreas in small amounts throughout the day (basal rate of insulin secretion). In addition, the amount of insulin secreted by the pancreas may be modified under certain circumstances. For example, the pancreas of a non-diabetic person normally secretes larger amounts of insulin (bolus rate of insulin secretion) when the person ingests a meal to prevent postprandial hyperglycemia, i.e., abnormally increased sugar content in the blood.

In contrast to the non-diabetic person, a diabetic person's pancreas may not secrete the required amount of insulin and/or the diabetic person's metabolism may become resistant to the insulin produced. Thus, the diabetic person has to somehow artificially introduce the insulin into the body. One method of introducing the insulin is by the conventional insulin formulation injection method using a syringe or other manual injection mechanism. Using this method, the body's blood glucose level may be monitored (for example, by checking a blood sample) and the amount of insulin to be injected may be adjusted accordingly. For example, after a meal the blood glucose level may be monitored and an appropriate amount of insulin may be injected into the bloodstream of the user.

In the alternative, a diabetic person may choose to use an infusion pump such as the infusion pump described above. By using an infusion pump, a continuous basal rate of insulin delivery is maintained with additional bolus injections of insulin delivered by the pump in accordance with the user's needs. These needs may be determined based on prior experience, the results of glucose monitoring (for example, by a sensing device in combination with a communication device).

While today's infusion pumps are actively controlled by the patient, previous art describes the concept of an artificial pancreas solution, consisting of a glucose sensor, a computer, and an infusion pump. As discussed above, a sensing device associated with the pump may monitor the blood glucose level of the user and the blood glucose level may then be used by the pump to automatically regulate the delivery of the insulin formulation.

It is known to use as a control device a process controller for performing automatic regulation of the infusion pump. The process controller, for example a processor or other computing element, controls the process such that a process variable is maintained at a desired set point value (also referred to as the “goal”). Such process controllers typically use a set of control parameters which have been determined through, for example, experimentation or calculation, to operate in an optimal manner to control the process variable. Although not the only possible technique, these control parameters are typically dependent on the anticipated range of differences (“error values”) that result between the process variable and the set point during actual operation of the process.

Ordinarily, infusion formulation delivery systems utilize control systems having an input-response relationship. A system input, such as a sensed biological state, produces a physiological response related to the input. Typically, the input (such as a sensed blood glucose level) is used to control some parameter associated with the response variable (such as an insulin infusion rate or an amount of insulin).

A process controller employed in the delivery of an insulin formulation typically executes a closed-loop algorithm that accepts and processes a blood glucose level input supplied to the controller by a sensing device. The closed-loop algorithm may adjust insulin formulation delivery as a function of, for example, the rate of change over time of the sensed glucose level.

These closed-loop algorithms have many limitations. Some of these limitations result from the fact that a process controller employing a closed-loop algorithm to control the delivery of an insulin formulation may be restricted to only adding insulin formulation to the system. Once insulin formulation is added to the system, normally the controller cannot retrieve it.

Additional limitations result from the fact that certain parameters affecting glucose production may not be adequately compensated for by these closed-loop algorithms. For example, certain daily events may significantly affect glucose production levels in the human body. Thus, these events may also significantly affect the amount of insulin required to metabolize the glucose.

Exercise, for example, has been shown to lower blood glucose levels in the human body. Thus, exercise may result in a dip in blood glucose levels and a corresponding decrease in the amount of insulin formulation delivered by the body. Longer or more strenuous exercise events may result in a greater dip in blood glucose level than shorter and less strenuous exercise events.

Similarly, sleep and stress may affect the body's ability to burn carbohydrates and therefore may affect glucose levels. For example, glucose metabolism has been found to be slower in a sleep deprived state. In addition, elevations of certain stress hormones within the body may also result in slower glucose metabolism. Thus, longer or shorter periods of sleep or stress may result in more or less significant changes in glucose levels.

Furthermore, the ingestion of certain medications may affect a user's sensitivity to insulin, i.e. a given amount of insulin may be more or less sufficient depending on whether or not a particular medication has been taken.

An additional event that may significantly affect the production of glucose in the body is the ingestion of food. This results in part from the fact that during digestion carbohydrates are broken down into glucose that then enters the bloodstream. In addition, the amount and type of foods ingested affect the amount of glucose produced. This may contribute to the finding that every year a number of insulin-using diabetic patients are found dead in bed, presumably from nocturnal hypoglycemic events.

For the reasons cited above, closed-loop algorithms employed for controlling delivery of an insulin formulation in response to sensed blood glucose levels are unlikely to adequately compensate for the affects such daily events may have on blood glucose levels. Thus, the diabetic person relying on such closed-loop algorithms may be at an increased risk of hypoglycemia and/or hyperglycemia. Moreover, the algorithm the ultimately does control glycemia in an adequate fashion must be tailored specifically to the individual person.

A closed loop algorithm for controllably providing measured programmed amounts of insulin to a person is disclosed in U.S. Pat. No. 6,740,072 to Starkweather, et al., the contents of which are incorporated herein by reference in its entirety, and where portions of the description of the prior art patent are reproduced herein in whole or in part to provide an understanding of this invention. The algorithm disclosed has proportional, derivative, and basal rate components which calculate a delivery amount of an infusion formulation such as insulin to a person. Control parameters are provided which may be adjusted in real time to compensate for changes in a sensed biological state that may result from daily events. Safety limits on the delivery amount may be included in the algorithm. The algorithm may be executed by a computing element within a process controller for controlling closed loop infusion formulation delivery. The biological state is sensed by a sensing device which provides a signal to a controller. The controller calculates an infusion formulation delivery amount based on the signal from the algorithm and sends commands to an infusion formulation delivery device which delivers an amount of infusion formulation determined by the commands.

In contrast to the Starkweather invention, it is the subject of this patent to disclose a highly individualized monitored pancreatic solution in which the input from a sensor or plurality of sensors relay input first to a local computer hub and from there to a remote computing/monitoring station that is able to compute an individualized algorithm for insulin administration and exert oversight over the local artificial pancreas solution in order to improve patient safety.

### SUMMARY OF THE DISCLOSURE

In an exemplary embodiment of the present invention, there is disclosed a monitored closed loop infusion formulation delivery system for controlling a biological state in the body of a user, comprising:
a sensor or plurality of sensors for measuring parameters of a sensed biological state at timed intervals and generating a signal representative of the measured parameters and times at which the measurements are taken;
a computing element for receiving and processing the generated signal, wherein the computing element adjusts control parameters within an algorithm to compensate for changes in the sensed biological state resulting from events affecting the sensed biological state;
calculates a delivery rate of an infusion formulation after adjusting the control parameters; and
generates commands based on the calculated delivery rate;
a delivery device for receiving the generated commands and delivering the infusion formulation based on the generated commands;
wherein the algorithm receives measured parameters and uses control parameters, the control parameters being different from the measured parameters, and
   wherein the algorithm is used for calculating an infusion formulation delivery rate; and
a remotely located monitoring station that communicates with the computing element via radio or wire to further adjust the control parameters within the algorithm to meet changing needs in the body of the user or to ensure the safety of the user if an out of protocol state is detected.

It is an advantage of the present invention to provide an external monitoring facility to oversee the closed-loop algorithm for controlling delivery of insulin formulation which may be adjusted in real time in a manner tailored to the unique circumstances of each individual in order to more accurately determine whether a blood glucose level is rising or falling over a predetermined interval.

It is a further advantage of the present invention to provide an external monitoring facility that may adjust the safety limits for bolus delivery that may be compared with samples of blood glucose parameters at predefined intervals of time and which enable or disable bolus delivery based on the comparisons.

It is a further advantage of the present invention to provide an external monitoring facility that may modify the safety limits on the amount of insulin formulation that may be stored in an accumulator during a predefined time interval.

It is a further advantage of the present invention to provide external emergency shutoff capability, the ability to sense a patient fall or other signs of distress, and the ability to geolocate the patient in order to dispatch emergency assistance.

It is a further advantage of the present invention to direct the delivery of a rescue drug to counteract the effect of the infused drug. In the case of diabetes, the rescue drug for insulin might be glucagon.

The limitations of the prior art closed loop systems previously described may, therefore, be surmounted via converting such systems to a “monitored pancreatic device,” through the addition of a long-range (i.e. cellular or other broadband technology) radio facility that links the monitored-closed-loop system to an external monitoring facility composed of human and nonhuman components. Components of the closed-loop algorithm calculate a present value of infusion formulation in a body as well as whether that value is rising or falling overall during a predefined time interval. The closed-loop algorithm includes an equation whose variables are programmable in real time. The variables may be used as control parameters which may be adjusted to adjust the algorithm to more accurately calculate the present value of infusion formulation in the body. The monitoring station oversees and adjusts the monitored-closed-loop algorithm in order to meet the patient's changing needs and in order to ensure patient safety if an out-of-protocol state is detected. The closes loop algorithm may also take into account a variety of sensors, such as those capable of monitoring activity, heart rate, food ingestion, and other biological parameters in order to improve the accuracy with which glucose is controlled by the insulin infusion.

Preferred embodiments of the present invention provide an otherwise closed-loop algorithm for use with a proportional-derivative controller for delivering an insulin formulation which comprises an equation for calculating a proportional component, a derivative component, and a basal component of an amount of insulin formulation to be delivered based on a sensed blood glucose level. The OCL algorithm is housed in the controller that links the sensor devices to the infusion pump. That controller is connected via long range radio to the monitoring station. Control parameters within the closed-loop algorithm may be programmable in real time and may be adjusted to compensate for events which may significantly affect the blood glucose level.

Depending upon the context of use, the invention may include various combinations of these features which function together to provide both adjustable control parameters and safety limits on the delivery of infusion formulation in response to a detected biological state.

The more important features of the invention have thus been outlined in order that the more detailed description that follows may be better understood and in order that the present contribution to the art may better be appreciated. Additional features of the invention will be described hereinafter and will form the subject matter of the claims that follow.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting. One of those alternative embodiments is the use of this system to direct the patient to inject insulin via manual means, such as with a syringe or pen. In a variation of this embodiment, the injection device may be linked by wire or wirelessly to the central hub in order to electronically set the volume of insulin to be injected and/or to automatically record that amount of insulin injected by the patient. In another alternative embodiment, the sensor may be limited to a traditional finger stick glucose meter, linked by wireless connection to the central monitoring station and providing the patient with direct treatment guidance from both human and computer sources.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

The foregoing has outlined, rather broadly, the preferred feature of the present invention so that those skilled in the art may better understand the detailed description of the invention that follows. Additional features of the invention will be described hereinafter that form the subject of the claims of the invention. Those skilled in the art should appreciate that they can readily use the disclosed conception and specific embodiment as a basis for designing or modifying other structures for carrying out the same purposes of the present invention and that such other structures do not depart from the spirit and scope of the invention in its broadest form.

### BRIEF DESCRIPTION OF THE DRAWINGS

Other aspects, features, and advantages of the present invention will become more fully apparent from the following detailed description, the appended claim, and the accompanying drawings in which similar elements are given similar reference numerals..

FIG. 1 shows a block diagram of an infusion formulation delivery system utilizing a control system having an external monitoring system which includes automated and human elements which may more accurately regulate an infusion formula delivery rate and which may be used to protect a patient's safety by affording an opportunity to manually alter insulin flow, shut off insulin flow, communicate with the patient, or in an emergency situation geolocate the patient and provide an emergency response in accordance with the principle of the invention;.

FIG. 2 shows a flow diagram of a general process performed by the closed-loop algorithm which is disclosed in U.S. Pat. No. 6,740,072 an which is incorporated herein in its entirety for adjusting infusion formulation delivery as a function of a change in a sensed biological state; and

FIG. 3 is a graph of a blood glucose response curve (on the y axis) as a function of time (on the x axis) of a typical human blood glucose response to the ingestion of a meal.

### DETAILED DESCRIPTION OF THE PREFERRED EMBODIMENT

For the purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated devices, and such further applications of the principles of the invention as illustrated therein being contemplated as would normally occur to one skilled in the art to which the invention relates.

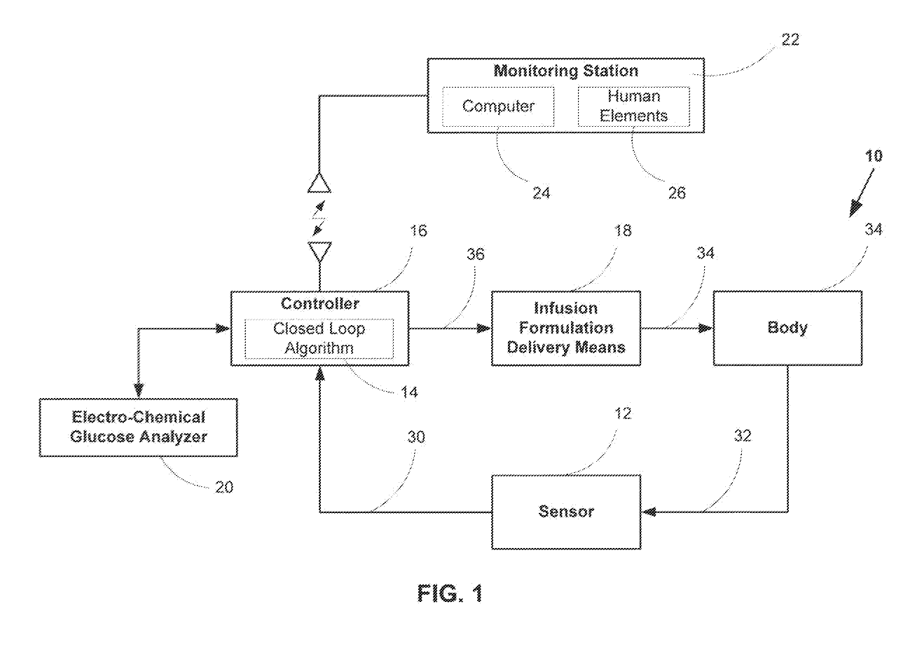

Referring to FIG. 1, there is disclosed a block diagram of an infusion formulation delivery system utilizing a control system having an external monitoring system which includes automated and human elements which may more accurately regulate an infusion formula delivery rate and which may be used to protect a patient's safety by affording an opportunity to manually alter insulin flow, shut off insulin flow, communicate with the patient, or, in an emergency situation, geolocate the patient and provide an emergency response.

As noted above the present invention relate to an infusion formulation delivery system 10 which includes a monitoring station coupled to an algorithm such as a closed loop algorithm for use with a process controller for controlling the delivery of an infusion formulation to a body based in part on a sensed biological state within the body and on information obtained by the monitoring station.

Continuing with FIG. 1, at least one biological sensor 12 that detects analyses and other physiological parameters serves as an input to an algorithm such as a closed loop algorithm 14 located within a controller 16. The connections between the at least one sensor 12 and the controller may be physical or wireless.

The closed loop algorithm 12 may be the closed loop algorithm which is more fully disclosed in detail in U.S. Pat. No. 6,740,072, the contents of which are incorporated herein by reference in its entirety. The closed loop algorithm 12 accurately calculates a delivery amount of an infusion formulation based on a sensed biological state and programmable control parameters. The algorithm calculates the delivery amount having proportional, derivative, and basal rate components. The control parameters may be adjusted in real time to compensate for changes in a sensed biological state that may result from daily events. Components of the closed-loop algorithm calculate a present value of infusion formulation in a body as well as whether that value is rising or falling overall during a predefined time interval and includes an equation whose variables are programmable in real time. The variables may be used as control parameters which may be adjusted to adjust the algorithm to more accurately calculate the present value of infusion formulation in the body. Safety limits on the delivery amount may be included in the algorithm. The algorithm may be executed by a computing element within a process controller for controlling closed loop infusion formulation delivery. The biological state is sensed by at least one sensing device which provides a signal to the controller 16 which calculates an infusion formulation delivery amount.

The controller is connected via a physical or wireless connection to an infusion formulation delivery means 18 such as an insulin delivery mechanism. The insulin delivery mechanism may be an infusion pump or manual infusion device, such as a pen injector. The controller unit 16 may include an electrochemical glucose analyzer 20 for the purposes of calibrating the system. A monitoring station 22 receives information from and sends information to the closed loop algorithm 14 in the controller 16 via a long range radio connection which may be cellular 2G, 3G, 4G, or a future cellular technology, or may also be WI-MAX or other WAN protocol in nature. The monitoring station 22 includes a computer 24 and human elements 26 which oversee and adjust the output of the monitored closed loop algorithm in order to meet the patient's changing needs and to ensure the patient safety, if in an out of protocol state, is detected.

In an embodiment of the invention, and in addition to the above, other optional elements of the invention that may serve to enhance its benefits to the user may include, A) Accelerometers and other sensors for measuring body activity in order to measure exercise and metabolism and to identify signs of distress such as lurching gait or falling down. B) Various sensors that may detect food ingestion and resulting metabolic effects, C) Sensors which may geolocate the patient and which may be valuable in dispatching an emergency response, and D) Links to outside laboratory values and electronic medical records which may enable the algorithm to incorporate variables not available within the closed loop system.

The embodiment of FIG. 1 of the invention may be used in conjunction with a delivery device such as an infusion pump which is utilized in an implant environment within a human body or in other biological implant or non-implant environments which, include but are not limited to external infusion devices, pumps or the like.

The infusion pump may be configured for delivery of an insulin formulation used to regulate glucose levels in a diabetic user. However, other embodiments may be employed in the delivery of other infusion formulations having other pharmacological properties.

Continuing with FIG. 1. sensor 12 generates a sensor signal 30 which is representative of a system parameter input 32 such as a blood glucose level of a human body 34, and sends the sensor signal 30 to the closed loop algorithm 14 located in the controller 16. The controller 16 receives the sensor signal 30 and generates command signals 36 that are communicated to the infusion formulation delivery device 18. The command signals are also sent to the monitoring station 22 which includes computer 24 and human elements 26 which oversee and adjust the output of the monitored closed loop algorithm in order to meet the patient's changing needs and to ensure the patient's safety if an out of protocol state is detected. The infusion formulation delivery device 18 then delivers the infusion formulation output 38 to the body 34 at a determined rate and amount in order to control the system parameter 32.

The at least one sensor 12 may comprise a sensor, sensor electrical components for providing power to the sensor and generating the sensor signals 30, a sensor communication system for carrying the sensor signal 30 to controller 16, and a sensor housing for enclosing the electrical components and the communication system. Controller 16 may include one or more programmable processors, logic circuits, or other hardware, firmware or software components configured for implementing the control functions, a controller communication system for receiving the sensor signal 30 from the at least one sensor 12, and a controller housing for enclosing the controller communication system and the one or more programmable processors, logic circuits, or other hardware, firmware or software components. The infusion formulation delivery device 18 may include a suitable infusion pump, infusion pump electrical components for powering and activating the infusion pump, an infusion pump communication system for receiving commands from the controller 16, and an infusion pump housing for enclosing the infusion pump, infusion pump electrical components, and infusion pump communication system.

The external monitoring system which includes the monitoring station 22 that has an automated computer 24 and human elements 26 to more accurately regulate the infusion formula delivery rate and which may be used to protect the patient's safety by affording an opportunity to manually alter insulin flow, shut off insulin flow, communicate with the patient, or in an emergency situation geolocate the patient and provide an emergency response. In addition, the external monitoring system oversees the operation of the closed loop algorithm for controlling delivery of insulin formulations which may be adjusted in real time to more accurately determine whether a blood glucose level is rising or falling over a predetermined interval. Still further the external monitoring system may modify the safety limits on the amount of insulin formulation that may be stored in an accumulator during a predefined time interval, provide emergency shutoff capability, sense that a patient has fallen or is experiences distress, or has the ability to geolocate a patient in order to dispatch emergency assistance. The monitoring station oversees and adjusts the closed loop algorithm in order to meet the patient's changing needs and ensure the patient safety if an out of protocol state is detected.

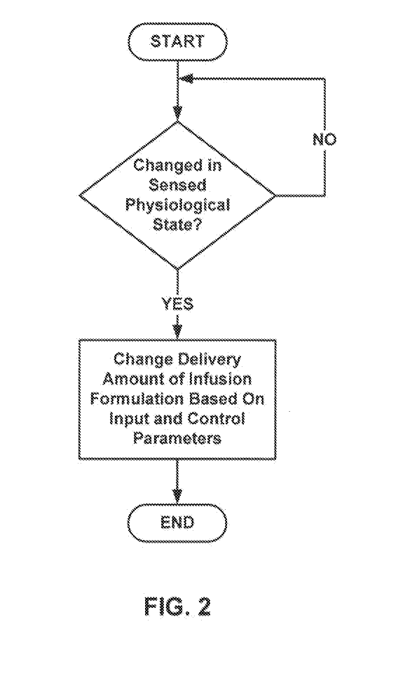

Referring to FIG. 2, there is shown a flow diagram of the general process performed by the closed-loop algorithm 14 which is disclosed in U.S. Pat. No. 6,740,072 and which is incorporated herein in its entirety for adjusting infusion formulation delivery as a function of a change in a sensed biological state where, for example, the rate of change is over time for a sensed biological state. At start, block 50, the program advances to block 52 where a check is made for a change in the biological state at timed intervals. A sensing device such as sensor 12 detects a change in glucose level and communicates the change directly to the closed loop algorithm in the controller 16. If no change, NO, is detected, the closed-loop algorithm loops back to block 52, and repeats this process until a change is detected. When a change occurs, YES, the closed-loop algorithm determines the amount and/or rate of infusion formulation required based on the input and various parameters that have been programmed into the controller, block 54, and the monitoring station will oversee and may adjust this amount and/or rate of infusion formulation.

Where the infusion formulation delivery system 100 shown in. FIG. 1 includes a controller 16 used for controlling an insulin response to a sensed blood glucose level, the closed-loop algorithm may be of the proportional-derivative (PD) type. The use of a PD type closed-loop algorithm is advantageous, for example, when processing resources such as processor power and/or memory may be limited. In an alternative embodiment, a proportional-integral-derivative (PID) type closed-loop algorithm may be used.

PD controllers may utilize a closed-loop algorithm which computes both a proportional component and a derivative component of a response (output) to changes in a system parameter (input). For example, the proportional and derivative components may be combined to calculate an amount of insulin formulation to be delivered in response to a present sensed blood glucose level (system parameter input 32) within a body 34. The controller may then issue commands 36 to, for example, output a calculated amount of insulin formulation (output 38) to an infusion site on or within the body 34 based on the present sensed blood glucose level.

As disclosed in the identified US Patent, the magnitude of each component's contribution to the calculated amount of insulin formulation to be delivered to the infusion site may be expressed by a formula or equations, such as the following equations:

U.sub.P=.alpha.(G.sub.(t)−G.sub.sp) Equation 1

and

U.sub.D=.beta.dG/dt, Equation 2

where
U.sub.P is the proportional component of the response,
U.sub.D is the derivative component of the response,
alpha. is a proportional gain coefficient,
beta. is a derivative gain coefficient,
G is a present blood glucose level,
G.sub.sp is a desired blood glucose level or “set point” for the blood glucose level, and
t is the time at which the blood glucose level is sensed.

There is a desired blood glucose level G.sub.sp for each person which may be determined, for example, from experimentation or from the person's historical physiological data. The closed-loop control system may be designed to maintain the desired blood glucose level G.sub.sp for a particular person. It may do this, in part, by measuring the difference between the determined G.sub.sp and a blood glucose level G sensed at time t (G.sub.(t)). This difference is the blood glucose level error at time t that must be corrected.

The proportional component expressed in Equation 1 determines whether the blood glucose level error is positive, negative, or zero, (i.e., whether G.sub.(t) is, respectively, higher, lower, or equal to G.sub.sp). Thus, G.sub.sp is subtracted from G.sub.(t). If G.sub.(t) is higher than G.sub.sp, the controller 16 may generate an insulin formulation delivery command 36 to drive the infusion formulation delivery device 18 to provide insulin formulation (output 38) to the body 34. If G.sub.(t) is lower than G.sub.sp, the controller 16 may reduce or stop delivery of the insulin formulation to the body 34 by the infusion formulation delivery device 18. The result of subtracting G.sub.sp from G.sub.(t) is then multiplied by a proportional gain coefficient .alpha.. The derivative component dG/dt expressed in Equation 2 determines if the blood glucose level is presently rising or falling and at what rate of change.

Thus, to determine the amount of infusion formulation to be delivered at any point in time (I.sub.(t)), the following standard equation may be used:

I.sub.(t)=.alpha.(G.sub.(t)−G.sub.sp)+.beta.dG/dt Equation 3

where I.sub.(t) is the amount of insulin formulation to be delivered based on the sensed blood glucose level at time t.

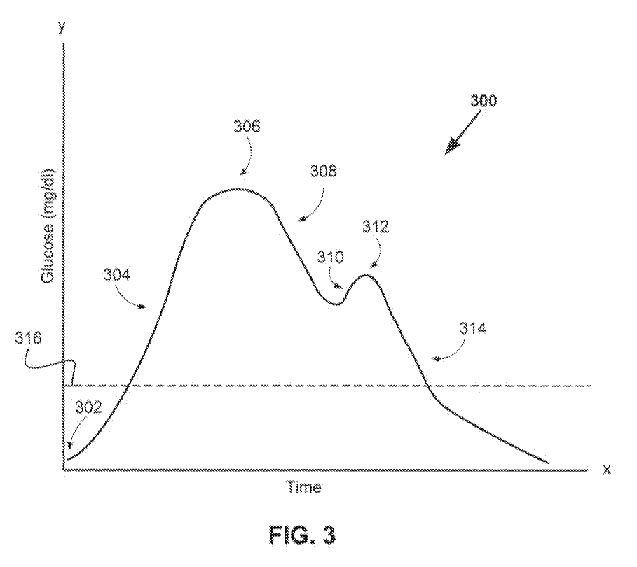

Referring to FIG. 3, there is shown a typical human blood glucose response curve 300 (on the y axis) as a function of time (on the x axis) to the ingestion of a meal. This blood glucose response curve 300 is representative of blood glucose levels sensed at various sampling times as a system parameter 32 by a sensor 12, as shown in FIG. 1. After a person ingests a meal 302, there is typically a steady rise 304 in blood glucose level over time until the blood glucose level reaches a peak 306. It has been observed from experimentation that peak 306 may occur approximately 90 minutes after ingestion of the meal. After peak 306 has been reached, it has been observed that the blood glucose level then begins to decrease 308 over time. During the decline from the first peak 306, a second temporary rise 310 in blood glucose level has been observed. A second peak 312 results from this temporary rise 310. This second peak 312 may occur approximately 30 to 90 minutes after the occurrence of peak 306 and typically tends to occur 30 to 60 minutes after the occurrence of peak 306.

After peak 312 has been reached, it has been observed that the blood glucose level then continues as before to decrease 314 over time. Although the reasons for this second, temporary rise 310 are not completely understood at the present time, it is a consistently observable phenomenon that presents a problem for a closed-loop algorithm.

To understand the problem, it is helpful to understand the response of a closed-loop algorithm at the various points of the response curve 300 shown in FIG. 3. As stated above, at point 302, the meal is ingested. As the blood glucose level rises 304 above the set point 316, a closed-loop algorithm may calculate both the amount by which the present blood glucose level exceeds the set point value (a proportional component) and may also determine that the blood glucose level is rising at a certain rate (a derivative component). Thus, a closed-loop algorithm may calculate a result based on these two components which causes a command to issue from a controller associated with the algorithm to deliver a calculated amount of insulin at a time t on the response curve 300 corresponding to 304.

At peak 306 of the response curve 300, the blood glucose level is neither rising nor falling, but the proportional component calculates that it is still above the set point and therefore the controller associated with the closed-loop algorithm may continue to issue commands to deliver more insulin formulation, although it may not be as large an amount as that issued at 304 on the response curve 300.

At 308, the proportional component calculates that the blood glucose level is still above the set point. However, now the blood glucose level is falling, and therefore the controller associated with the closed-loop algorithm may issue commands to deliver a decreased amount of insulin formulation based on the calculation of the derivative component.

At 310, the proportional component calculates that the blood glucose level is still above the set point. The derivative component will calculate that the blood glucose level is rising again. At this point, the controller associated with the closed-loop algorithm may issue a command to deliver another significant amount of insulin based on this information although, seen globally, the blood glucose level is decreasing overall. Thus, because of this additional input of insulin formulation into the system, the risks of hypoglycemia to the user are increased.

The closed loop algorithm which is disclosed in the referenced U.S. Patent and used in this invention addresses the limitations of a closed-loop algorithm exemplified above in relation to FIG. 3 more accurately determine the amount of insulin formulation to be delivered based on a sensed blood glucose level by including programmable control parameters which may be used to introduce discontinuities in the calculation of I.sub.(t) unlike the continuous calculations of I.sub.(t) performed by the closed-loop algorithm described above.

The closed loop algorithm in combination with the monitoring station which oversees and adjusts the monitored closed loop algorithm in order to meet the patient's changing needs may be more effective at maintaining a desired blood glucose level for a particular user under circumstances where blood glucose level may be significantly affected by events such as, but not limited to meals, sleep, and exercise. As a result, the risk of hypoglycemia and/or hyperglycemia in the user may be reduced.

In some instances the derivative component of the closed-loop algorithm (dG/dt) shown in Equation 2 above is referred to as the “trend term” and may be expressed, as:

Trend term=(G.sub.(t)−G.sub.(t−x)) Equation 4

where x is a numerical value representing an increment of time.

The value of the trend term may be calculated at predetermined intervals, for example each minute, and is used to determine the “trend” of G, i.e., whether the value of G is trending up or trending down during a timeframe determined by the term (t−x). Thus, by changing the value of x, where the value of x may be programmable, the timeframe for sampling the trend may be lengthened or shortened. As an example, using Equation 4, if x=10 minutes, the blood glucose level sensed 10 minutes prior in time to time t is subtracted from the blood glucose level sensed at time t.

Generally, a shorter timeframe (and, thus, a smaller value of x) is preferred for trend calculation because the shorter the timeframe, the more responsive the infusion formulation delivery system may be to a rising or falling blood glucose level. However, this responsiveness must be balanced against noise susceptibility of the sensor signal, which may increase as the timeframe gets shorter. After the trend term is calculated, it is multiplied by the derivative gain coefficient .beta.

The proportional gain coefficient a and derivative gain coefficient .beta. (.where beta. is also referred to as the “trend gain”) may be chosen based, for example, on experimentation. As an example, they may be chosen based on observations of the insulin response of several, normal glucose tolerant users. An average of the values of these responses may then be taken. Alternatively, other statistical values besides an average value may be used, for example a maximum or minimum value, standard deviation value, or some other suitable value.

In some instances both the proportional and derivative gain coefficients may be programmable. In addition, .beta. may be programmed as one value when the trend is going up and a different value when the trend is going down (also referred to as the “trend up” and “trend down” gains).

It is believed that even if G.sub.(t) is equal to G.sub.sp (in other words if the proportional component of the response is zero), a certain minimal amount of insulin formulation should still be delivered in order to maintain that condition. Thus, in addition to Equation 1 and Equation 2 shown above, a basal insulin formulation delivery amount is included as a further component of the response. This basal component (B.sub.0) represents a minimum amount of insulin formulation that would be delivered when G.sub.(t) is equal to or greater than G.sub.sp (i.e., when the blood glucose level at time t is equal to or greater than the desired blood glucose level or set point) and without regard to the rate at which the blood glucose level is rising or falling and B.sub.0 may be programmable and may be selected from a programmable table of multiple B.sub.0 values based on certain criteria. By selecting B.sub.0 values from the programmable table, different values of B.sub.0 may be selected for different parts of the day (for example, dawn). Thus, different parts of the day may be treated differently than other parts of the day.

Thus, to determine the amount of infusion formulation to be delivered at any point in time (I.sub.(t)) the following equation may be used by embodiments of the present invention:

I.sub.(t)=.alpha.(G.sub.(t)−G.sub.sp)+.beta.((G.sub.(t)−G.sub.(t−x))/x)+−B.sub.0

Equation 5.

After a meal has been ingested by a user, the amount of insulin formulation to be delivered based on a sensed blood glucose level may be more accurately determined by establishing, for example from historical physiological data, a time window within which the temporary rise in blood glucose level occurs in the user. Once this time window has been established, embodiments of the present invention may disable any further commands from issuing from the controller (for example, commands 36 from controller 16 in FIG. 1), by, for example, programming start and stop times for the time window that may be used by the controller to suspend any further calculations of I.sub.(t) during the time window.

It can be seen from FIG. 7 of the referenced US Patent that because the second rise 710 and resulting second peak 712 occur within the programmed time window, the second rise does not result in any increase in delivered insulin formulation. This discontinuity in the calculation of I.sub.(t) may thus cause I.sub.(t) to be calculated based only on the global downward trend of response curve 700. Therefore, the temporary rise 710 does not cause any increase in the amount of delivered insulin formulation, and the risk of hypoglycemia to the user is reduced.

In further embodiments of the present invention, the amount and/or rate of delivered insulin formulation may modified based on inputs from sensing devices that detect other biological states in lieu of or in addition to the sensed blood glucose level. For example, it has been observed that a user's blood oxygen levels may change based on whether the user is awake or sleeping. As noted above, sleep is an event which may significantly affect blood glucose levels in particular users. Thus, the blood oxygen level of a user may be sensed to determine if the user is asleep and this information is input to the closed-loop algorithm in order to adjust the amount and/or delivery rate of insulin formulation.

Similarly, it has been observed that body temperature may significantly affect blood glucose levels. Thus, a temperature sensor which monitors body temperature may include this information as an input to the controller in order to adjust the amount and/or delivery rate of insulin formulation.

The closed loop algorithm may include a sensing device for detecting whether or not a user is exercising. An accelerometer or other device suitable for detecting motion may be used to detect motion as an indicator of current physical activity. Exercise may significantly affect blood glucose levels in particular users. Thus, information from an exercise sensing device may be input to the controller in order to adjust the amount and/or delivery rate of insulin formulation based on this information.

Referring again to FIG. 1, sensor 12 may sense many biological states including, but not limited to, blood, glucose level, blood oxygen level, and temperature. Sensor 12 may further include an exercise sensing device such as an accelerometer. a separate blood glucose level sensor, blood oxygen level, temperature sensor and exercise sensing device or sensors that detect various combinations of these and/or other biological states.

An infusion pump for the delivery of an infusion formulation may have a fixed pump stroke volume, i.e., there is a certain minimum value of infusion, formulation that must be accumulated before a pump stroke is executed, which is referred to as a “pump stroke volume.” Thus, if I.sub.(t) is calculated on a periodic basis, for example each minute, then the calculated amount for each minute may be some fractional part of a pump stroke volume. These fractional parts may be stored, for example, in a chamber or reservoir within or adjacent to the infusion pump until an amount equal to the pump stroke volume has been accumulated. At that time, a pump stroke may be executed and the insulin formulation delivered.

A large amount of insulin formulation (a “bolus”) may be delivered by the infusion formulation delivery device, independently of Equation 5, when a user has a blood glucose level that is above a predefined value and is rising at or above a predefined rate, thus possibly indicating that a meal has been consumed. In other words, when the predefined criteria is met, the bolus amount may be delivered instead of a value of I(t) calculated using Equation 5.

Predefined bolus safety limits are included as control parameters for the closed-loop algorithm and the bolus control parameters may be programmable in real time.

Thus, bolus safety limits are provided to reduce the possibility of erroneously delivering a bolus by ensuring that predefined conditions for delivery of a bolus are met by testing predefined control parameters that are programmable. Thus, the closed-loop algorithm reduces the possibility of delivering too much insulin formulation as a bolus and thus reduces the risks of hypoglycemia to the user.

Similarly, a limit may be set on the maximum amount of insulin formulation that may be delivered by the infusion formulation delivery device in one hour. This amount may also be programmable.

Thus, by “clamping” the maximum amount that may be stored in the accumulator at each sampling period and the maximum amount that may be delivered to the body each hour, the possibility of delivering too much insulin formulation is reduced and the risks of hypoglycemia to the user is reduced.

Accordingly, by combining the closed-loop algorithm with the monitoring station which oversees and adjusts the closed loop algorithm, the closed-loop algorithm may more accurately determine an amount of insulin formulation to be delivered in response to a sensed blood glucose level in order to reduce the risks of hypoglycemia to a user. Additional aspects and features of the closed-loop algorithm may provide safety limits which reduce the risks of hypoglycemia to a user.

While there have been shown and described and pointed out the fundamental novel features of the invention as applied to the preferred embodiments, it will be understood that the foregoing is considered as illustrative only of the principles of the invention and not intended to be exhaustive or to limit the invention to the precise forms disclosed. Obvious modifications or variations are possible in light of the above teachings. The embodiments discussed were chosen and described to provide the best illustration of the principles of the invention and its practical application to enable one of ordinary skill in the art to utilize the invention in various embodiments and with various modifications as are suited to the particular use contemplated All such modifications and variations are within the scope of the invention as determined by the appended claims when interpreted in accordance with the breadth to which they are entitled.

What is claimed is:

1. A closed loop infusion formulation delivery system for controlling a biological state in the body of a user, comprising:
a sensor or plurality of sensors for measuring parameters of a sensed biological state at timed intervals and generating a signal representative of the measured parameters and times at which the measurements are taken;
a computing element for receiving and processing the generated signal, wherein the computing element adjusts control parameters within an algorithm to compensate for changes in the sensed biological state resulting from events affecting the sensed biological state;
calculates a delivery rate of an infusion formulation after adjusting the control parameters; and
generates commands based on the calculated delivery rate;
and
a delivery device for receiving the generated commands and delivering the infusion formulation based on the generated commands;
wherein the algorithm receives measured parameters and uses control parameters, the control parameters being different from the measured parameters, and
wherein the algorithm is used for calculating an infusion formulation delivery rate; and
a remotely located monitoring station that communicates with the computing element via radio or wire to further adjust the control parameters within the algorithm to meet changing needs in the body of the user or to ensure the safety of the user if an out of protocol state is detected.

2. The closed loop infusion formulation delivery system recited in claim 1, wherein the infusion formulation comprises an insulin formulation and wherein the sensed biological state comprises blood glucose levels in a human body.

3. The closed loop infusion formulation delivery system recited in claim 2, wherein the control parameters are programmable.

4. The closed loop infusion formulation delivery system recited in claim 3, wherein the control parameters are programmable in real time.

5. The closed loop infusion formulation delivery system recited in claim 2, wherein the control parameters comprise at least one of a glucose set point, basal rate, proportional gain, trend term, trend up gain, and trend down gain.

6. The closed loop infusion formulation delivery system recited in claim 1, wherein the measured parameters of the sensed biological state comprise a present blood glucose level and a rising or falling rate of change for the blood glucose level.

7. The closed loop infusion formulation delivery system recited in claim 1, wherein the sensed biological state comprises one of a sensed blood oxygen level, a temperature, or motion.

8. The closed loop infusion formulation delivery system recited in claim 7, wherein the infusion formulation comprises an insulin formulation.

9. The closed loop infusion formulation delivery system recited in claim 1, wherein the sensor comprises a sensor for measuring at least one of a blood glucose level, a blood oxygen level, a temperature, or motion.

10. The closed loop infusion formulation delivery system recited in claim 1, wherein the sensor comprises a two or more sensors, each of the two or more sensors measuring at least one of a blood glucose level, a blood oxygen level, a temperature, or motion.

11. The closed loop infusion formulation delivery system recited in claim 1, wherein the radio transmission between the closed loop and the monitoring station is either GSM or CDMA.

12. The closed loop infusion formulation delivery system recited in claim 1, wherein the radio transmission between the closed loop and the monitoring station is protected by 128 bit or higher encryption.

13. A method of providing a closed loop infusion formulation delivery system for controlling a biological state in the body of a user, comprises:
providing a sensor for measuring parameters of a sensed biological state at timed intervals and generating a signal representative of the measured parameters and times at which the measurements are taken;
providing a computing element for receiving and processing the generated signal,
wherein the computing element adjusts control parameters within an algorithm to compensate for changes in the sensed biological state resulting from events affecting the sensed biological state;
calculates a delivery rate of an infusion formulation after adjusting the control parameters; and
generates commands based on the calculated delivery rate;
and
providing a delivery device for receiving the generated commands and delivering the infusion formulation based on the generated commands;
wherein the algorithm receives measured parameters and uses control parameters, the control parameters being different from the measured parameters, and
wherein the algorithm is used for calculating an infusion formulation delivery rate; and
providing a remotely located monitoring station that communicates with the computing element via radio or wire to further adjust the control parameters within the algorithm to meet changing needs in the body of the user or to ensure the safety of the user if an out of protocol state is detected.

14. The closed loop infusion formulation delivery system recited in claim 13, wherein the infusion formulation comprises an insulin formulation and wherein the sensed biological state comprises blood glucose levels in a human body.

15. The closed loop infusion formulation delivery system recited in claim 14, wherein the control parameters are programmable.

16. The closed loop infusion formulation delivery system recited in claim 15, wherein the control parameters are programmable in real time.

17. The closed loop infusion formulation delivery system recited in claim 14, wherein the control parameters comprise at least one of a glucose set point, basal rate, proportional gain, trend term, trend up gain, and trend down gain.

18. The closed loop infusion formulation delivery system recited in claim 13, wherein the measured parameters of the sensed biological state comprise a present blood glucose level and a rising or falling rate of change for the blood glucose level.

19. The closed loop infusion formulation delivery system recited in claim 13, wherein the sensed biological state comprises one of a sensed blood oxygen level, a temperature, or motion.

20. The closed loop infusion formulation delivery system recited in claim 19, wherein the infusion formulation comprises an insulin formulation.

21. The closed loop infusion formulation delivery system recited in claim 13, wherein the sensor comprises a sensor for measuring at least one of a blood glucose level, a blood oxygen level, a temperature, or motion.

22. The closed loop infusion formulation delivery system recited in claim 13, wherein the sensor comprises two or more sensors, each of the two or more sensors measuring at least one of a blood glucose level, a blood oxygen level, a temperature, or motion.
